(19) United States
(12) Patent Application Publication
(10) Pub. No.: US 2019/0208800 A1
(43) Pub. Date: Jul. 11, 2019

(54) L-GLUTAMINE IN SWINE DIETS

(71) Applicant: The United States of America, as represented by the Secretary of Agriculture, Washington, DC (US)

(72) Inventor: Jay S. Johnson, WEST LAFAYETTE, IN (US)

(21) Appl. No.: 16/224,886

(22) Filed: Dec. 19, 2018

Related U.S. Application Data
(60) Provisional application No. 62/613,823, filed on Jan. 5, 2018.

(51) Int. Cl. A23K 20/142; A23K 50/30; A61K 31/198

(52) U.S. Cl. CPC A23K 20/142; A23K 50/30; A61K 31/198; A61P 37/02; A23V 2200/31; A23V 2200/324; A23V 2250/062; A23V 2002/00

(57) ABSTRACT

A swine feed may include a non-antibiotic immunomodulator, for example L-glutamine. The use of such a diet may include feeding it to piglets post-weaning and transportation to alleviate some of the negative effects of such stressors on pig production and welfare. Use of L-glutamine in the piglets' diet may have several beneficial effects, such as the reduction of enteric disease and faster weight gain. The use of L-glutamine may be a cost-effective replacement for traditional dietary antibiotics in swine production.

### BACKGROUND

In commercial swine production systems, newly weaned piglets are often subjected to multiple stressors in early life (e.g., transportation between 4 and 24 hours to grow-finish facilities, weaning, temperature stress, etc.) during which time weight loss, dehydration and disease states are exacerbated. To alleviate the effects of these stressors, traditional practices call for the inclusion of dietary antibiotics (ex. 400 g chlortetracycline and 35 g tiamulin/ton of feed) in the diet for 14-21 days post-weaning and transport in order to reduce pathogenic bacterial loads and help promote recovery and improve growth rates of piglets.

Consumers of animal products are becoming increasingly concerned with the use of antibiotics in animal husbandry largely due to fears regarding antibiotic resistance and residual antibiotics in the carcass, thus placing the U.S. livestock industry under immense pressure to eliminate or reduce antibiotic programs and search out cost-effective alternatives. In fact, a recent 2017 Veterinary Feed Directive banned the use of growth promoting dietary antibiotics in swine production. In addition, some niche markets such as organic producers, the U.S. poultry industry, and livestock producers in the European Union have banned the use of prophylactic antibiotics in animal feed. Although some antibiotic alternatives have been developed (probiotics, prebiotics, organic acids, etc.), many alternatives are either not effective and/or can be cost-prohibitive in commercial production systems.

There is a need for cost-effective antibiotic alternatives that improve productivity and promote recovery similarly to traditional dietary antibiotic programs following traditional production stressors in livestock species.

All of the references cited herein, including U.S. patents and U.S. patent application Publications, are incorporated by reference in their entirety.

Mention of trade names or commercial products in this publication is solely for the purpose of providing specific information and does not imply recommendation or endorsement by the U.S. Department of Agriculture.

### SUMMARY

According to a first aspect of the invention, a composition of swine feed may include a therapeutically effective amount of a non-antibiotic immunomodulator.

According to a further aspect of the invention, the non-antibiotic immunomodulator may also be a growth promotant.

According to a further aspect of the invention, the non-antibiotic immunomodulator may be L-glutamine.

According to a further aspect of the invention, the non-antibiotic immunomodulator may be present in an amount of about 0.10%-1.0% (w/w) as compared to the total amount of the swine feed.

According to yet a further aspect of the invention, the non-antibiotic immunomodulator may be present in an amount of about 0.20%-0.60% (w/w) as compared to the total amount of the swine feed.

According to another aspect of the invention, a method of decreasing negative effects of a stressor on swine may include providing the swine with a first swine feed, the first swine feed including a therapeutically effective amount of a non-antibiotic immunomodulator, wherein the swine is provided with the first swine feed during a period before, during, or after the stressor is introduced to the swine.

According to a further aspect of the invention, the stressor may be one of weaning and transportation.

According to a further aspect of the invention, the swine may be provided with the first swine feed during a period of at least 14 days after the stressor is introduced.

According to a further aspect of the invention, the negative effects may be the incidence of enteric disease in the swine.

According to a further aspect of the invention, the method may also include providing the swine with a second swine feed, wherein the second swine feed does not contain the non-antibiotic immunomodulator, and wherein the swine may be provided with the first swine feed during a treatment period, and after the treatment period elapses the swine is provided with the second swine feed during a growth period.

According to a further aspect of the invention, the treatment period may be as long desired, for example about 14 days in length.

Further, the use of L-glutamine may be more cost-effective (reduces feed costs for producers by 18-20%) than traditional dietary antibiotic programs and may be more effective in improving productivity, promoting recovery, and reducing therapeutic antibiotic injections compared to traditional dietary antibiotics (based on reduced percentage of piglets treated for illness, increased feed intake and greater growth rate) following traditional production stressors (i.e., weaning and transport).

### BRIEF DESCRIPTION OF THE FIGURES

Advantages of embodiments of the present invention will be apparent from the following detailed description of the exemplary embodiments. The following detailed description should be considered in conjunction with the accompanying figures in which:

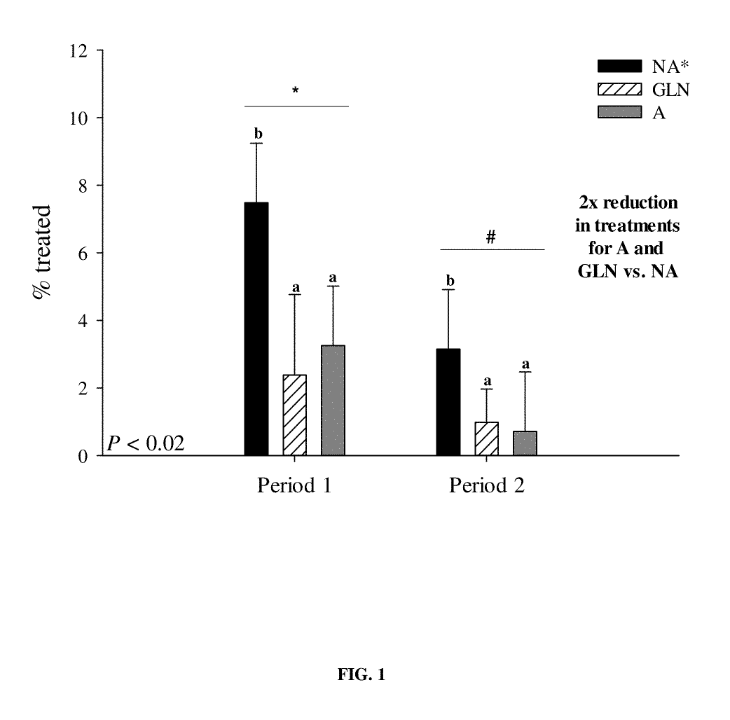

Exemplary FIG. 1 shows effects of post-weaning and transport dietary supplementation of antibiotics (A), L-glutamine (GLN), and no antibiotics (NA) on treatment for enteric disease in swine. Period 1 is 0-14 days post-weaning and transport and Period 2 is 15-34 days post-weaning and transport. Error bars represent ±1 standard error of the mean (SEM). Letters (a, b) indicate statistically significant differences (P≤0.05) between dietary treatments and symbols (*, #) indicate statistically significant differences (P≤0.05) between periods.

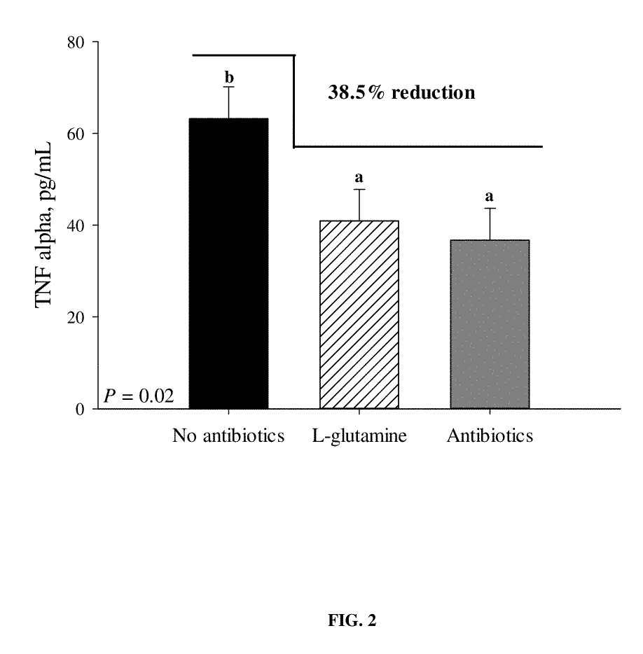

Exemplary FIG. 2 shows the effects of A, GLN, and NA diets on post-weaning and transport pigs with regard to tumor necrosis factor alpha (TNFα) in the pigs' blood. Error bars represent ±1 standard error of the mean (SEM). Letters (a, b) indicate statistically significant differences (P≤0.05) between dietary treatments.

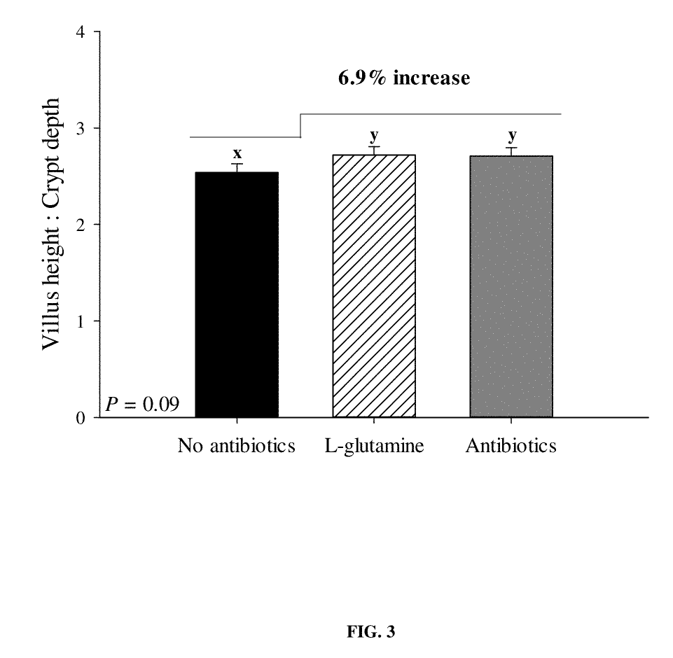

Exemplary FIG. 3 shows the effects of A, GLN, and NA diets on post-weaning and transport pigs with regard to the ratio of villus height to crypt depth in the intestines of the pigs. Error bars represent ±1 standard error of the mean (SEM). Letters (x, y) indicate statistical tendencies (0.05<P≤0.10) between dietary treatments.

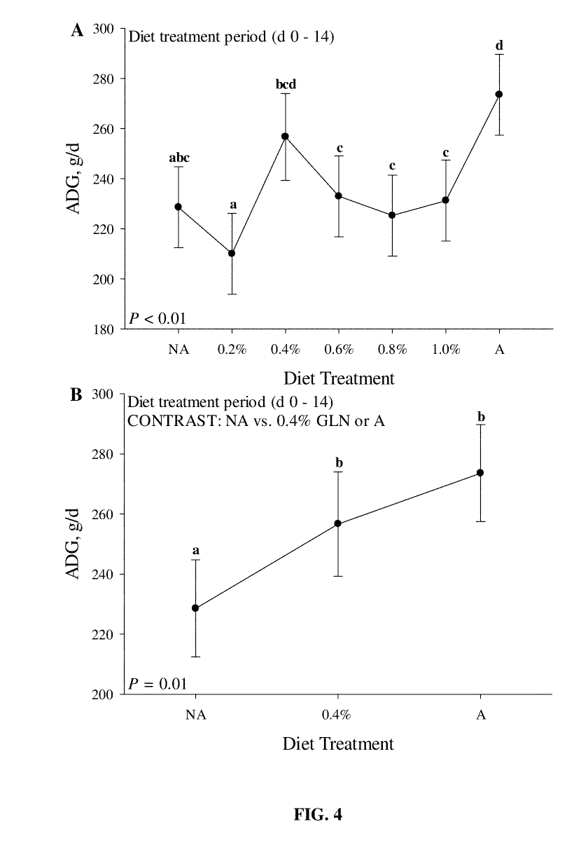

Exemplary FIG. 4 shows the effects of A, GLN, and NA diets, where the GLN diets may be 0.2% L-glutamine (GLN-02), 0.4% L-glutamine (GLN-04), 0.6% L-glutamine (GLN-06), 0.8% L-glutamine (GLN-08), or 1.0% L-glutamine (GLN-10), on post-weaning and transport pigs. In particular, average daily gain (ADG) is shown. Error bars represent ±1 standard error of the mean (SEM). Letters (a, b, c, d) indicate statistically significant differences (P≤0.05) between dietary treatments.

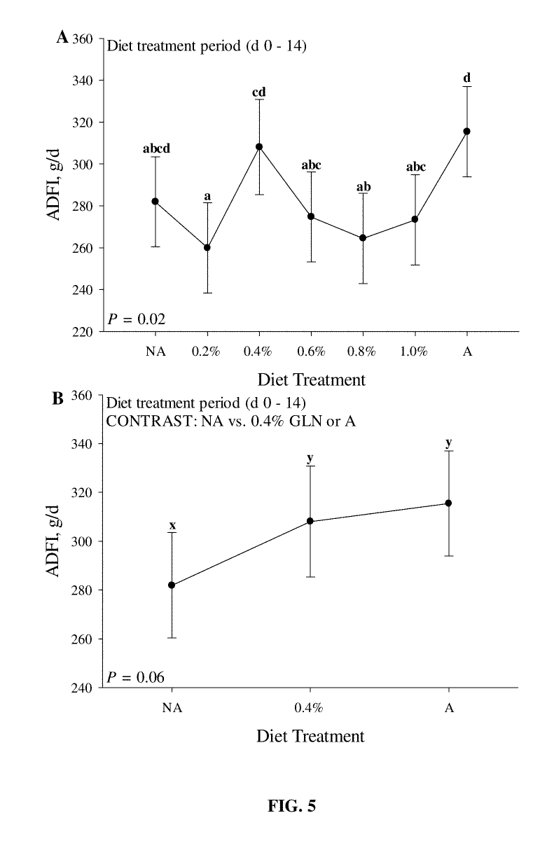

Exemplary FIG. 5 shows the effects of NA, GLN-02, GLN-04, GLN-06, GLN-08, GLN-10, and A diets on post-weaning and transport pigs with respect to average daily feed intake (ADFI). Error bars represent ±1 standard error of the mean (SEM). Letters (a, b, c, d) indicate statistically significant differences (P≤0.05) between dietary treatments. Letters (x, y) indicate statistical tendencies (0.05<P≤0.10) between dietary treatments.

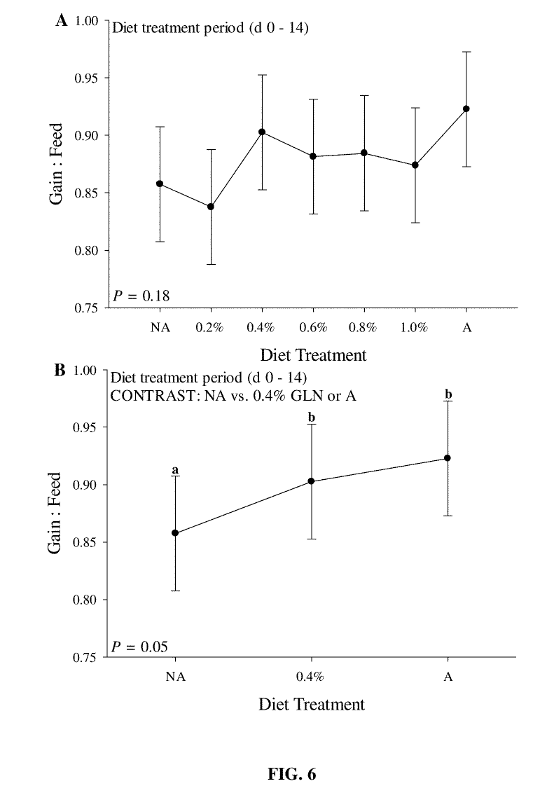

Exemplary FIG. 6 shows the effects of NA, GLN-02, GLN-04, GLN-06, GLN-08, GLN-10, and A diets on post-weaning and transport pigs with respect to feed efficiency (Gain: Feed). Error bars represent ±1 standard error of the mean (SEM). Letters (a, b) indicate statistically significant differences (P≤0.05) between dietary treatments.

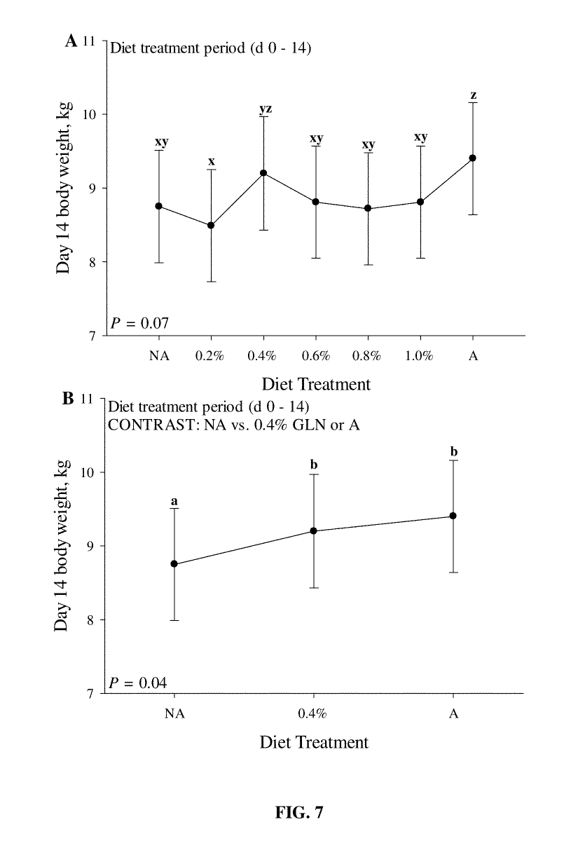

Exemplary FIG. 7 shows the effects of NA, GLN-02, GLN-04, GLN-06, GLN-08, GLN-10, and A diets on post-weaning and transport pigs with respect to body weight after 14 days on the diet. Error bars represent ±1 standard error of the mean (SEM). Letters (a, b) indicate statistically significant differences (P≤0.05) between dietary treatments. Letters (x, y, z) indicate statistical tendencies (0.05<P≤0.10) between dietary treatments.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Unless defined otherwise, all technical and scientific terms used herein have the same meaning as commonly understood by one of ordinary skill in the art to which the invention belongs. As used herein, the term “about” refers to a quantity, level, value, or amount that varies by as much as 30%, preferably by as much as 20%, and more preferably by as much as 10% to a reference quantity, level, value, or amount. Although any methods and materials similar or equivalent to those described herein can be used in the practice or testing of the present invention, the preferred methods and materials are now described.

Other compounds may be added to the composition provided they do not substantially interfere with the intended activity and efficacy of the composition; whether or not a compound interferes with activity and/or efficacy can be determined, for example, by the procedures utilized below.

The amounts, percentages, and ranges disclosed herein are not meant to be limiting, and increments between the recited amounts, percentages, and ranges are specifically envisioned as part of the invention.

“Optional” or “optionally” means that the subsequently described event or circumstance may or may not occur, and that the description includes instances in which said event or circumstance occurs and instances where it does not. For example, the phrase “optionally comprising X” means that the composition may or may not contain X, and that this description includes compositions that contain and do not contain X.

The term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein.

The term “antibiotic” refers to an antibacterial agent which inhibits the growth of or kills microorganisms. As used herein, an antibiotic is explicitly not an immunomodulator.

The term “immunomodulator” refers to a chemical or biological agent which modifies the immune response of an organism or the functioning of the immune system of an organism. As used herein, the term “immunomodulator” only refers to such agents which improve the functioning of the immune system of the organism.

The term “swine” refers to animals of the family Suidae, and is used interchangeably herein with the term “pigs.”

The term “growth promotant” is synonymous with “growth promotor.” The immunomodulator of the present invention may function as solely an immunomodulator or as both an immunomodulator and a growth promotant, depending on biological variables.

According to at least one exemplary embodiment of the invention, a composition of swine feed may include a therapeutically effective amount of a non-antibiotic immunomodulator. The non-antibiotic immunomodulator may be, for example, L-glutamine. The swine feed containing the non-antibiotic immunomodulator may be fed to pigs before, during, and/or after a stressor is introduced to the pigs.

### Example 1

In a small-scale example, three groups of post-weaning pigs were subjected to a simulated transport experience immediately post-weaning and then provided different diets to assess the effects of L-glutamine as a supplement in the diet. The details of the procedure are given in full in “Evaluating the behavior, growth performance, immune parameters, and intestinal morphology of weaned piglets after simulated transport and heat stress when antibiotics are eliminated from the diet or replaced with L-glutamine” by Johnson, J. S. and Lay Jr., D. C. in the Journal of Animal Science (2017, 95, 91-102), which is incorporated herein in its entirety by reference.

In this experiment, the piglets were fed on disparate feeds for 14 days post-weaning and simulated transport, and then euthanized by CO₂ exposure and exsanguination. The feeds were formulated as shown in Table 1:

TABLE 1
Diet formulations (as-fed basis)

| Ingredient | Percent (%) |
|---|---|
| Corn | 37.295 |
| Soybean Meal, 48% | 19.000 |
| Monocal. Phosphate | 0.510 |
| Limestoen | 0.580 |
| Salt | 0.250 |
| Soybean oil | 5.000 |
| Lysine-HCl | 0.250 |
| DL-Methionine | 0.225 |
| L-Threonine | 0.120 |
| L-Tryptophan | 0.020 |
| Swine Vit. Premix | 0.250 |
| Swine TM Premix | 0.125 |
| Selenium premix | 0.050 |
| Dried whey | 25.000 |
| Sel. Men. Fish Meal | 4.000 |
| Plasma protein | 2.500 |
| SD Blood Meal | 1.250 |
| Soy Concentrate | 2.500 |
| Zinc Oxide | 0.375 |
| Phytase (600 PU/g) | 0.100 |
| Rabon Larvacide | 0.025 |
| Treatment premix¹ | 0.575 |

¹The treatment premix consisted of fine ground corn for the no-antibiotic diet; Aureomycin (110 g chlortetracycline/kg) and Denagard (22 g/kg tiamulin) for the antibiotic diet; and 0.20% L-glutamine (remainder fine ground corn) for the L-glutamine diet

Overall, the L-glutamine group showed better productivity compared to the non-antibiotic and antibiotic group. Other results showed that the L-glutamine was approximately as effective as the antibiotics in other parameters as well.

### Example 2

In a larger example, three groups of post-weaning and post-transport pigs (each group n=160, total of 480 pigs, 50% male and 50% female) were fed on different diets to assess the effects of L-glutamine as a supplement in the diet in a production environment. The diets consisted primarily of corn and soybean meal as in Example 1. One group (Group A) was fed a standard swine feed in two phases containing a standard antibiotic supplement (400 g chlortetracycline and 35 g tiamulin/ton of feed), another group (GLN) was fed a standard swine feed in two phases containing 0.20% L-glutamine, and a third group (NA) was given a standard swine diet in two phases containing no antibiotics or immunomodulators. Otherwise, the diets for groups A, GLN, and NA were the same. The disparate diets were given to the different groups for 14 days post-weaning and transport, following which all three groups were on a common diet for the remainder of the trial.

This experiment was conducted from Jul. 28, 2016 to Sep. 21, 2017. The pigs were followed all the way through market (i.e., day 125 post-weaning, being about 6 months of age).

FIG. 1 shows the percentage of pigs treated in each of two time periods, Period 1 and Period 2. Period 1 represents days 0-14 post-weaning and transport and Period 2 represents days 15-34 post-weaning and transport. The measure of the y-axis is the percentage of each group treated for enteric diseases by farm staff who were blind to diet content. Swine farm staff are trained to identify pigs exhibiting signs of enteric disease as pigs with diarrhea and weight loss and lethargy (i.e. hanging head, lying down, and refusal to eat). Data were analyzed on a per pen basis and the assumptions of normality of error, homogeneity of variance, and linearity were confirmed post-hoc. Because these data were not normally distributed they were log transformed to meet assumptions of normality prior to analysis in SAS 9.4. For ease of interpreting the data, the data were then back-transformed for presentation in graph form and shown in FIG. 1.

One of the primary goals of providing piglets with dietary antibiotics is as an enteric disease preventative to reduce the need to administer additional treatments for illness. FIG. 1 shows that piglets provided L-glutamine had the same requirement for injectable therapeutic antibiotics for enteric disease as the piglets fed the antibiotics, and this continued even after the dietary treatments ceased.

Furthermore, growth data at the nursery stage (d0-d34 post-weaning) was analyzed to ensure that L-glutamine does not negatively affect the growth of the piglets. These data are shown in Table 2 below:

TABLE 2
Overall nursery growth performance in pigs (d
0 to d 34 post-weaning) provided antibiotics (A),
L-glutamine (GLN), and no antibiotics (NA).

| | Dietary Treatment | | | | P- |
|---|---|---|---|---|---|
| Parameter | A | GLN | NA | SE | value |

d 0 to 14

| | | | | | |
|---|---|---|---|---|---|
| Initial body weight, kg | 5.58 | 5.59 | 5.57 | 0.29 | 1.00 |
| Average daily gain, g | 224.23^{a} | 210.78^{a} | 189.24^{b} | 10.19 | 0.01 |
| Average daily feed intake, | 277.1^{a} | 272.06^{a} | 253.63^{b} | 13.21 | 0.04 |
| g | | | | | |
| Feed efficiency, gain: feed | 0.84^{a} | 0.79^{ab} | 0.77^{b} | 0.01 | 0.01 |
| d 14 body weight, kg | 8.65^{a} | 8.50^{a} | 8.19^{b} | 0.26 | 0.01 |

d 15 to 34

| | | | | | |
|---|---|---|---|---|---|
| Average daily gain, g | 458.01 | 447.36 | 436.69 | 12.05 | 0.21 |
| Average daily feed intake, | 702.26 | 680.43 | 669.56 | 22.81 | 0.16 |
| g | | | | | |
| Feed efficiency, gain: feed | 0.65 | 0.66 | 0.65 | 0.01 | 0.78 |

d 0 to 34

| | | | | | |
|---|---|---|---|---|---|
| Average daily gain, g | 364.50^{a} | 352.73^{ab} | 337.71^{b} | 10.18 | 0.01 |
| Average daily feed intake, | 532.20^{x} | 517.08^{xy} | 503.19^{y} | 17.43 | 0.09 |
| g | | | | | |
| Feed efficiency, gain: feed | 0.73^{a} | 0.71^{ab} | 0.70^{b} | 0.01 | 0.03 |
| d 34 body weight, kg | 17.76^{a} | 17.49^{ab} | 16.96^{b} | 0.74 | 0.04 |

Letters (^{a,b}) indicate differences (P ≤ 0.05) within a row
Letters (^{x,y}) indicate tendencies (0.05 ≤ P ≤ 0.10) within a row

As can be seen in Table 2, during the diet treatment period (d 0-14 post-weaning), the L-glutamine and antibiotic groups had a significantly higher feed intake, average daily gain, and final body weight compared to the non-antibiotic group. During the common diet period (d 15-34 post-weaning) the differences in productivity were less pronounced; however, there was a numerical trend for the antibiotic and L-glutamine pigs to be more productive than the non-antibiotics pigs. Overall (d 0-34 post-weaning), the L-glutamine pigs performed similarly to the antibiotic pigs during the nursery phase when taking into account both the diet treatment (d 0-14 post-weaning) and the common diet (d 15-34 post-weaning) phases.

In addition, a decrease in enteric disease for both groups (FIG. 1) likely contributed to the improved productivity. However, because antibiotics also improves feed efficiency that is likely why their productivity was a bit higher than the L-glutamine group (as indicated by the numerically greater gain:feed). Nevertheless, the improvement in productivity for the GLN group above that of the NA group is significant, especially when scaled up to a large swine operations that are feeding thousands of pigs at one time.

As stated above, the pigs were followed through to market-readiness at six months of age. Table 3 below shows the overall growth performance in the pigs from the beginning of the grow-finish phase (d 0) until market (d 125 post-weaning):

TABLE 3
Overall grow-finish growth performance in pigs (d 0
to d 125 of the grow-finish phase) provided antibiotics
(A), L-glutamine (GLN), and no antibiotics (NA).

| | Dietary Treatment | | | | P- |
|---|---|---|---|---|---|
| Parameter | A | GLN | NA | SE | value |

d 0 to 62

| | | | | | |
|---|---|---|---|---|---|
| Initial body weight, kg | 17.78^{a} | 17.49^{a} | 16.96^{b} | 0.74 | 0.04 |
| Average daily gain, g | 0.78 | 0.76 | 0.76 | 0.01 | 0.32 |
| Average daily feed intake, | 1.80 | 1.76 | 1.75 | 0.03 | 0.40 |
| g | | | | | |
| Feed efficiency, gain: feed | 0.45 | 0.46 | 0.45 | 0.01 | 0.80 |
| d 62 body weight, kg | 65.99 | 65.02 | 64.31 | 0.96 | 0.22 |

d 62 to 125

| | | | | | |
|---|---|---|---|---|---|
| Average daily gain, g | 0.88 | 0.89 | 0.90 | 0.02 | 0.41 |
| Average daily feed intake, | 2.87 | 2.91 | 2.90 | 0.05 | 0.72 |
| g | | | | | |
| Feed efficiency, gain: feed | 0.30 | 0.31 | 0.31 | 0.01 | 0.17 |

d 0 to 125

| | | | | | |
|---|---|---|---|---|---|
| Average daily gain, g | 0.83 | 0.83 | 0.83 | 0.01 | 0.95 |
| Average daily feed intake, | 2.33 | 2.33 | 2.32 | 0.03 | 0.97 |
| g | | | | | |
| Feed efficiency, gain: feed | 0.38 | 0.38 | 0.38 | 0.01 | 0.54 |
| Final body weight, kg | 122.77 | 121.73 | 122.34 | 1.23 | 0.83 |

Letters (^{a,b}) indicate differences (P ≤ 0.05) within a row

As can be seen in Table 3, the L-glutamine dietary treatment was effective in improving the growth performance of piglets through the end of the nursery phase. This is reflected by the greater body weight compared to no antibiotic pigs at the start of the grow-finish phase. However, it appears that the impact on growth performance ended during the grow-finish phase. This is unsurprising as all pigs were on a common dietary treatment from d 15 to d 125 post-weaning. Had they been on the dietary treatment the increase in growth performance would likely have continued.

### Example 3

In a similar experiment to the one above in Example 2, an L-glutamine-containing diet was evaluated for pigs to determine how it affected their general health and productivity. The experiment was conducted under commercial production conditions using a total of 480 mixed-sex (50% male and 50% female) pigs. Pigs were weaned and then transported for 12 hours to the nursery facility. Once pigs arrived at the nursery facility they were provided 1 of 3 separate diets, those being no antibiotics or L-glutamine (NA); 0.20% L-glutamine (GLN); or dietary antibiotics (A) for 14 days. After the 14-day period all pigs were provided the same antibiotic-free diets until they reached market weight. Blood and intestinal samples were collected from pigs to determine whole body immune function and intestinal integrity.

The data presented in FIG. 2 show that the L-glutamine treated pigs had a similar whole body inflammatory response as pigs provided antibiotics. Specifically, blood samples from the pigs were tested for tumor necrosis factor alpha (TNFα), a pro-inflammatory cytokine, elevated levels of which can be indicative of systemic inflammation and immune system activation which can be detrimental to the health and productivity of pigs. A reduction in circulating TNFα would be indicative of a decreased whole body inflammatory response in pigs. Because TNFα is reduced at a similar level in pigs fed antibiotics and L-glutamine, this indicates that L-glutamine can reduce inflammation in pigs similarly to dietary antibiotics under commercial swine production conditions. In addition, these data correspond well with the reduction in the rate of injectable treatments for disease observed in the antibiotic and L-glutamine fed pigs compared to the no antibiotic fed pigs that was previously shown. Furthermore, because immune system activation is energetically costly, the decreased activation as indicated by the reduced tumor necrosis factor alpha may help explain the improvement in growth parameters of pigs fed L-glutamine and antibiotics compared to no antibiotics.

The data presented in FIG. 3 show that morphological indicators of intestinal health are improved at a similar level in L-glutamine and antibiotic fed pigs compared to no antibiotic fed pigs. In particular, intestinal samples from pigs were analyzed to determine the ratio of villus height to crypt depth in the intestines. Villus height to crypt depth ratio is a morphological measure of intestinal health and a greater ratio indicates an improvement in intestinal health. Because this ratio was greater in GLN and A fed pigs compared to those fed no antibiotics (NA) this indicates that L-glutamine could improve morphological indicators of intestinal health at a similar level as antibiotics under commercial swine production conditions. Improved intestinal health is essential for nutrient absorption and preventing pathogens from entering the body. Because this morphological indicator of intestinal health is improved in the L-glutamine fed pigs, this may imply that L-glutamine can improve the surface area for nutrient absorption which can lead to greater growth rates and improved productivity as indicated in the nursery growth performance table in Example 2 above. In addition, improved intestinal barrier function can reduce pathogen infiltration into the body leading to reduced rates of illness in newly weaned and transported pigs as indicated by the decrease in treatment for disease.

### Example 4

A further experiment was undertaken to determine the effect of different amounts of L-glutamine in the swine diet. The experiment was conducted under commercial production conditions using a total of 336 mixed-sex (50% male and 50% female) pigs. Pigs were weaned and then transported for 12 hours to the nursery facility. Once pigs arrived at the nursery facility they were group housed (n=6 pigs/pen) and provided 1 of 7 separate diets including: no growth promoting antibiotics or supplemental L-glutamine (NA); 0.20% L-glutamine (GLN-02); 0.4% L-glutamine (GLN-04); 0.6% L-glutamine (GLN-06); 0.8% L-glutamine (GLN-08); 1.0% L-glutamine (GLN-10); or dietary growth promoting antibiotics (A; 400 g chlortetracycline and 35 g tiamulin/ton of feed) for 14 days. After the 14-day period all pigs were provided the same antibiotic free diets until day 35 post-transport. From day 0 to day 35 post-transport, body weight and feed intake was determined every 7 days to evaluate average daily body weight gain (ADG), average daily feed intake (ADFI), and feed efficiency (Gain:Feed).

The data presented in FIGS. 4-7 demonstrate the effects of supplementing L-glutamine at different levels. In each case, the A frame shows the range of all supplementations, and since supplementation at 0.4% produced the best results in each case, the B frame shows a comparison of only NA, A, and GLN-04 for clarity. P-values were separately calculated for each variable measured (ADG, ADFI, Gain:Feed, and Day 14 weight).

Table 4 below provides an overview of all growth performance data from the 35-day experiment. Data within Table 4 are broken down into the dietary treatment period (day 0-14), the common diet period (d 15-35), and the overall data combining the dietary treatment and common diet periods (day 0-35).

TABLE 4
Overall growth performance in pigs (d 0 to d 35 of post-transport)
provided antibiotics (A), L-glutamine at 0.2%, 0.4%, 0.6%, 0.8%, and
1.0% (GLN-02, -04, -06, -08, and -10), and no antibiotics (NA).

| | Dietary Treatment | | | | | | |
|---|---|---|---|---|---|---|---|
| Parameter | NA | GLN-02 | GLN-04 | GLN-06 | GLN-08 | GLN-10 | A |
| d 0 to 14 | | | | | | | |
| Initial body weight, kg | 5.63 | 5.63 | 5.66 | 5.63 | 5.64 | 5.63 | 5.66 |
| ADG, g | 228.57 | 210.08 | 256.67 | 233.00 | 225.29 | 231.23 | 273.53 |
| ADFI, g | 281.93 | 259.92 | 308.09 | 274.75 | 264.51 | 273.39 | 315.48 |
| Gain:Feed | 0.86 | 0.84 | 0.90 | 0.88 | 0.88 | 0.87 | 0.92 |
| d 14 body weight, kg | 8.75 | 8.49 | 9.20 | 8.81 | 8.72 | 8.81 | 9.40 |
| d 15 to 35 | | | | | | | |
| ADG, g | 405.51 | 388.72 | 422.35 | 399.47 | 443.06 | 416.20 | 417.27 |
| ADFI, g | 608.35 | 577.99 | 639.82 | 621.27 | 663.76 | 636.59 | 653.96 |
| Gain:Feed | 0.67 | 0.68 | 0.66 | 0.64 | 0.67 | 0.66 | 0.64 |
| d 0 to 35 | | | | | | | |
| ADG, g | 334.74 | 317.27 | 355.86 | 332.88 | 355.95 | 342.21 | 359.77 |
| ADFI, g | 477.78 | 450.76 | 508.44 | 482.66 | 504.06 | 491.31 | 518.57 |
| Gain:Feed | 0.74 | 0.74 | 0.75 | 0.74 | 0.76 | 0.74 | 0.75 |
| Final body weight, kg | 17.26 | 16.66 | 18.04 | 17.20 | 18.17 | 17.55 | 18.16 |

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1: A composition of swine feed comprising a therapeutically effective amount of a non-antibiotic immunomodulator.

2: The composition of claim 1, wherein the non-antibiotic immunomodulator is also a growth promotant.

3: The composition of claim 1, wherein the non-antibiotic immunomodulator is L-glutamine.

4: The composition of claim 1, wherein the non-antibiotic immunomodulator is present in an amount of about 0.10%-1.0% (w/w) as compared to the total amount of the swine feed.

5: The composition of claim 4, wherein the non-antibiotic immunomodulator is present in an amount of about 0.20%-0.60% as compared to the total amount of the swine feed.

6: A method of decreasing negative effects of a stressor on swine, the method comprising:
providing the swine with a first swine feed, the first swine feed comprising a therapeutically effective amount of a non-antibiotic immunomodulator,
wherein the swine is provided with the first swine feed during a period before, during, or after the stressor is introduced to the swine.

7: The method of claim 6, wherein the non-antibiotic immunomodulator is also a growth promotant.

8: The method of claim 6, wherein the non-antibiotic immunomodulator is L-glutamine.

9: The method of claim 6, wherein the stressor is one of weaning and transportation.

10: The method of claim 6, wherein the swine is provided with the first swine feed during a period of at least 14 days after the stressor is introduced.

11: The method of claim 6, wherein the negative effects are the incidence of enteric disease in the swine.

12: The method of claim 6, further comprising providing the swine with a second swine feed,
wherein the second swine feed does not contain the non-antibiotic immunomodulator, and
wherein the swine is provided with the first swine feed during a treatment period, and after the treatment period elapses the swine is provided with the second swine feed during a growth period.

13: The method of claim 12, wherein the treatment period is about 14 days in length.
